(19) United States
(12) Patent Application Publication
(10) Pub. No.: US 2011/0160545 A1
(43) Pub. Date: Jun. 30, 2011

(54) ELECTRONIC INVESTIGATIVE DEVICE AND METHOD FOR IDENTIFYING THE TRUTH

(76) Inventor: Raman Mukundan Champadi, Bangalore (IN)

(21) Appl. No.: 12/995,186

(22) PCT Filed: May 30, 2008
(86) PCT No.: PCT/IN08/00344

§ 371 Date: Nov. 30, 2010

(51) Int. Cl. A61B 5/00

(52) U.S. Cl. 600/300

(57) ABSTRACT

The present invention provides a novel electronic investigative device for identification of truth from individuals who have committed an act of offence. The invention also provides a method for identification of truth based on bio-electric responses that are elicited by presentation of a unique design of nestled probes. The method advantageously utilizes the experiential knowledge present in a subject's brain that elicits a bio electric response to the presentation of probe. The invention is completely non-invasive and does not require an active participation of the subject. Further, the method is fully automated to enable tamper proof results.

### FIELD OF INVENTION

The present invention generally relates to the field of cognitive neuroscience. More particularly the present invention relates to the field of forensic investigation wherein embodiments of the invention relates to a method and device for identification of truth in human subjects, the said identification comprising of extraction of brain signatures.

### BACKGROUND AND PRIOR ART

Cognitive neuroscience has made very significant inroads into research of brain—mind phenomenon using various techniques such as functional neuroimaging of the brain, cognitive electrophysiology and neuropsychological tests. Of these, cognitive electrophysiology offers the most objective and easy to test method, in which the subject can participate and be in the test for long period with no discomfort.

Further cognitive electrophysiology offers highly tested and reliable methods of analysis, which are also used in many other signal-processing applications. Despite the wealth of information available about the functioning of brain behavior relationship using analysis of electrical properties of the brain, there has been hardly any attempt made to use the techniques for a purpose such as detection of experiential knowledge of past deeds and/or actions in an individual. Several studies using functional electrophysiology and clinical neuropsychological testing have shown that recall of autobiographic information is different from knowing or recognizing the entities in the environment and different brain structures are involved in the two functions

Recording and analysis of electrical oscillations have been in use since several decades for diagnostic and research purpose in neurology and neuroscience disciplines. Bio-Electrical activity has been used for clinical applications wherein the methods used are still based on visual qualitative and quantitative analyses, despite the fact that computerized recording are in vogue.

For research in neuroscience involving human and animal studies, signal processing using energy level transformations and time domain analyses have been widely used. There is a functional significance associated with different frequency ranges obtained by the change of the electrical oscillations. The corresponding time domain changes are called evoked potential or event related potential. All these analyses require the user to set the various parameters for both acquisition and analyses. Although signal processing and the related analyses are performed by computer, they are to be initiated and controlled by manually set controls. The data thus obtained must be exported to a database for any further statistical analysis by a independent statistical system.

The only alternate commercially available and documented system is called “brain fingerprinting”, which uses three cephalic channels from the midline of the brain. The three cephalic channels are used for recording an event related (time domain) activity called the P300, which is a neural evoked potential component of the electroencephalogram (EEG) and is associated with event of familiarity and/or novelty detection. In forensic investigations, the suspect, a human subject, is presented information in the visual and/or the auditory mode using privileged information known exclusively to the perpetrator and/or information relevant to the crime, known to many. The privileged information when presented on a computer monitor elicits a P300 response distinctly different from P300 elicited in others who have no knowledge of the existence of the privileged information. The significant P300 response elicited in presence of the privileged information is detected by the perpetrator. However, the P300 method involves active participation of the subject by physically responding to the statements, questions, or images.

Research on the neural correlates of remembrance of experience or autobiographical information shows that the areas in the brain activated during such remembrance is distinctly different from the areas activated during recognition of familiarity or during the process of knowing the external world. Remembrance of experiences results in the activation of the Anterior Cingulate Cortex and the Limbic System of the brain, whereas knowing results in the activation of Dorsolateral Prefrontal Cortex. The electrical oscillation profiles can differentiate the two functional systems and the profile can be used to detect the type of activation evoked by a probe presented to a subject. Changes in the activity levels of different frequency ranges have been found to be associated with different phases of cognitive processing.

For example, desynchronization of 8-12 Hertz (Hz) activity is seen during the presentation of any stimulus to the brain. The desynchronization is accompanied by increase in 13-34 Hz activity and their distribution over the brain is associated with encoding or semantic processing of the stimulus. A change in the activity in the range 1-4 Hz occurs during attending to internal processing while an increase in 5-8 Hz activity represents activation of hippocampal structures associated with accessing autobiographic information. Similarly, an increase in the phase linked activity in 35-45 Hz and 55-85 Hz are associated with neural binding required during recruitment of various brain areas for synchronized information processing. An increase in the fast activities in the posterior areas is also associated with the presence of mental visual imageries important for the remembrance of experiences. Similar increases in the fast activities in the central areas are associated with the activation of motor cortex, which may in turn represent motor mental imageries during remembrance.

Changes in the bioelectrical activity recorded from the surface of the head is carried out at both the frequency and time domain levels. Frequency domain analysis is carried out by using Fast Fourier Transformation while the time domain analysis is carried out by detecting the amplitudes and latencies of positive and negative deflections.

There is no other method available for detecting the presence of Experiential Knowledge in the brain of the individual. The P300 method, patented by Lorenz Farwell is based on the detection of familiarity by an individual and it can be sued only so long as the object used for detection remains unfamiliar to others. Even a person witnessing a crime will produce the same type of P300 findings as the perpetrator. Unfamiliarity to familiarity is a changing cognitive state and all those who get access to the privileged information produces the same type of P300 results.

There is a real need for using techniques, which will help investigating agencies to give up third degree methods during interrogation of suspects. Crime scenarios have changed considerably that no physical evidences are left behind in the crime scene, which could help investigators to proceed with investigation of the case. Information must be taken out from suspects, who have either colluded with those and or actually taken part in the crime. Polygraph analyses of physiological functions are in vogue for detection of a lie in a suspect. A person who tells a lie is expected to have conflict with the knowledge of reality while telling, which manifests as physiological changes such as those seen in the heart rate and blood pressure. Habituation and mental state justifying the acts prevent such physiological responses making the test less accurate. Further, the responses are seen only if the subject is made to respond to questions. The present invention overcomes the disadvantages of current analysis in that the test does not measure awareness of the reality or the actions committed by the individual, which is an automatic and spontaneous response in the brain. Instead the test measures the changes in the electrical responses of brain of the subject when he becomes aware of the actions committed. The changes related to awareness are absent if the person has not committed the act at all.

### SUMMARY OF THE INVENTION

The present invention provides a device for identifying truth from individuals who have committed an act from those who have not committed the act. More particularly, the invention provides a method and device for forensic investigation that assists in identification of individuals who have been involved in criminal acts as well as identifying individuals who have not been involved in suspected crimes. The invention provides a method for identification of truth through incorporation of nestled probe technique. Further the technique utilizes presence of visual mental imageries, motor mental imageries, action and responses, emotional states and intentions arranged in sequential manner with contextually accurate references. The invention also provides an integrated system for data acquisition, signal processing and statistical analyses of the data acquired.

### BRIEF DESCRIPTION OF DRAWINGS

FIG. 1 depicts the Block diagram of the Neuro Signature System (NSS) and the various parts.

FIG. 2 depicts a tetris map of an individual's response to the various stimulus provided as a set of sequences

Table 1 shows a sample sheet showing the response of the subject in correlation with the presentation of the sequence probes.

Table 2 shows a sample sheet of response tracking in case of (a) many subjects being tested for a predetermined set of probes and (b) a single subject being tested by presentation of probes at different occasions.

### DETAILED DESCRIPTION OF THE INVENTION

The present invention generally relates to the field of cognitive neuroscience. More particularly the present invention relates to the field of forensic investigation wherein embodiments of the invention relates to a method and device for identification of truth in human subjects, the said identification comprising of noninvasive means of extraction and analyses of brain signatures obtained from an individual through detection of changes in functional states in presence of experiential knowledge.

Further the method of the said invention comprises of:
Collation of Information
Creation and validation of Probes
Probe presentation and Data acquisition.
Signature extraction

Each of the steps mentioned above will be explained in detail as exemplary embodiments of the present invention the advantages of which will further be explained as an example of the present invention.

Collation of Information: In this step all the information regarding the subject and the information that needs to be verified is collated. Both the facts to be verified and the issues to be investigated are documented.

### Descriptions of Exemplary Embodiments

Exemplary Probes: Probes are designed using the information collected. A probe herein refers to a verbal statement and/or a visual image presented to the subject in a controlled environment. The verbal (auditory) probe presented comprises of first person statements and the visual probe comprises of images related to the information being processed.

Further the various visual and/or auditory probes designed are classified as
Neutral Probes—Probes that are unconnected to the current test such as factual information about the shape of an object like that of around ball or the value of a simple mathematical problem like that of addition as in 2+2
Control Probes—Probes related to personal life events of the subject, which are known to be true. The experiences are non-controversial and truthful and agreed upon by the investigating officer (IO) and the subject. The IO must certify them as events that really took place in the life of the subject. The events could be related to personal life, family, marriage, education, medical history. Experiential knowledge elicited by control probes validates the findings of the Target probes.
Target A Probes—Probes directly related to the test being carried out, based on the information supplied by the investigating agency and designed for eliciting experiential knowledge
Target B Probes—Probes directly related to the test being carried out based on the information supplied by the subject and designed for eliciting experiential knowledge

These probes are utilized in designing of the nestled probes which are an essential part of the test used for eliciting the bioelectric signatures. The concept of nestled probes is that of contextual specificity in terms of time, space, sequence of action contents, and relevance of the information provided in each probe. The probes designed as mentioned earlier cover the crime scenario and other activities as considered appropriate by the investigating agency and as proposed by the subject.

Further the probes designed utilizing the information described earlier are coded using the code list made for the test, which depicts the sensory, motor, proprioceptive, emotional and awareness content to be evoked by each probe, which is recognized by the analysis system. The duration of the probe and its presentation are controlled by the system the working of which will be described in detail below. The system detects the functional state of the brain of the subject and interprets it as appropriate for receiving the probe. Table 1 shows the response of the subject to the probe sequence presented in a given scenario. A plurality of the said probe sequence maybe presented to the subject wherein the said sequence of probe maybe an auditory and/or visual probe. Further there may be a plurality of such scenarios wherein a plurality of probe sequence may be presented.

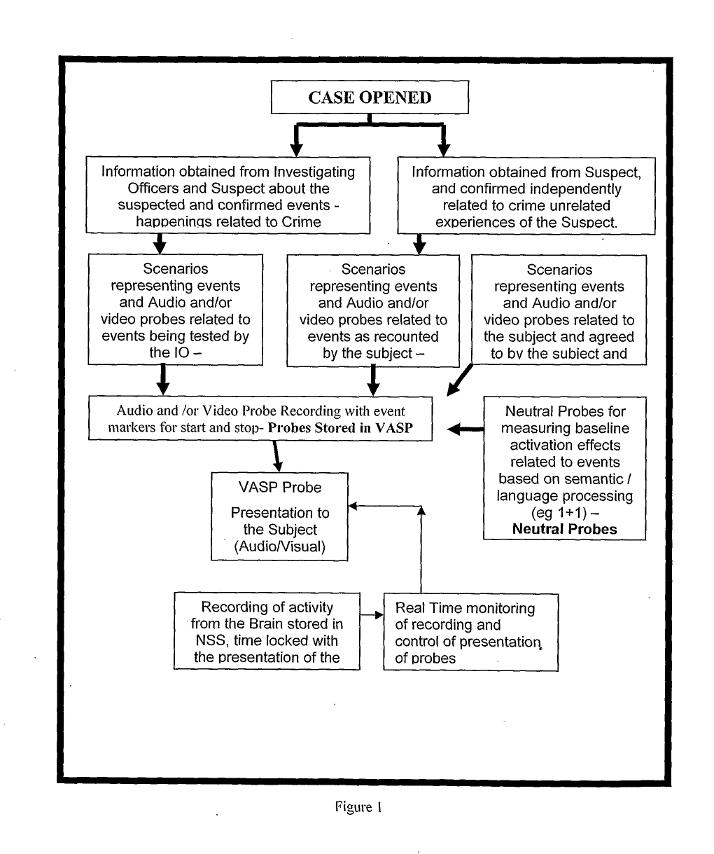

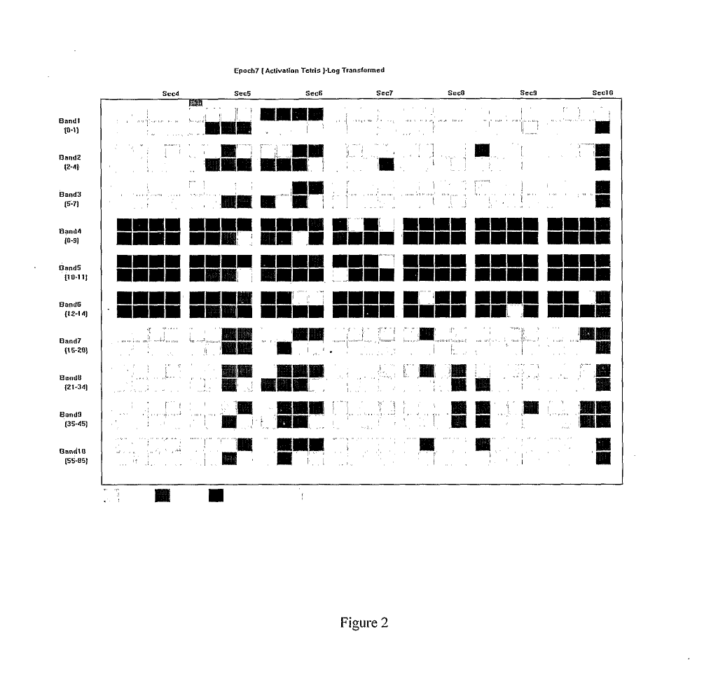

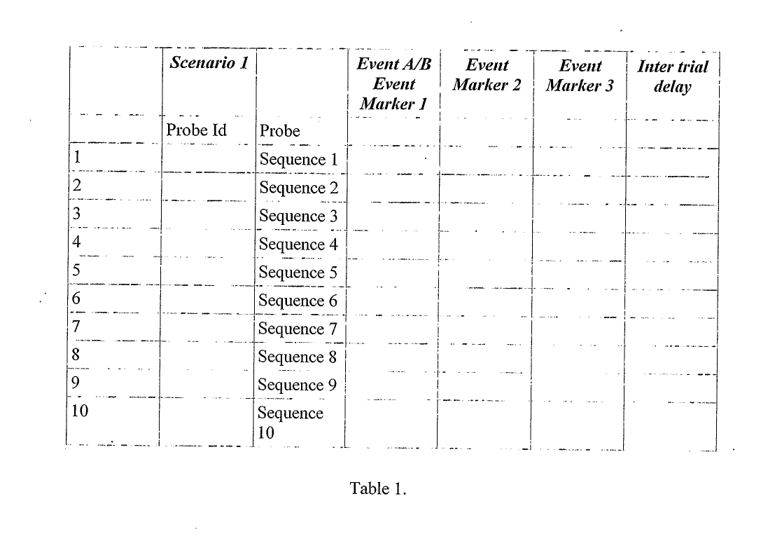

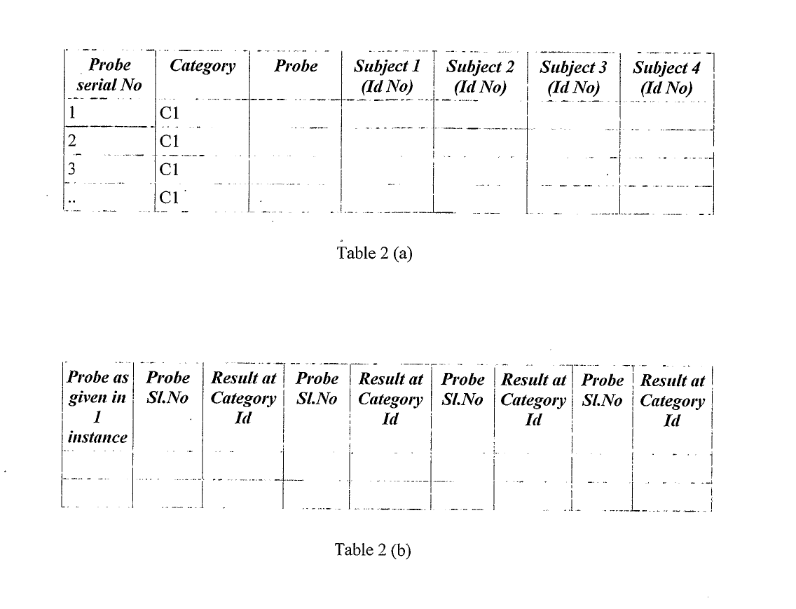

Probe presentation: The probe presentation unit is represented by Unit 5 of FIG. 1 and is termed as Visual and Auditory Stimulus Presentation Unit (VASP). VASP is responsible for presenting visual and auditory stimulus to the subject. It's operation is controlled by Unit 4 indicated in FIG. 1 which performs functions such as when to start the test, which visual and auditory stimulus to present, when to present the stimulus and stopping the test. The unique feature of the VASP include:
Control of the probe presentation to the subject
Synchronization of probe presentation and brain activation.
Enabling scientists a view of the probes being presented.

Exemplary Neuro Signature System (NSS): FIG. 1 shows the schematic block diagram of the system utilized for Data acquisition and analyses, referred to herein as NSS. Embodiments of the NSS are specifically designed for capturing the bioelectrical signatures obtained by presentation of probes to the subject, storage of the signatures obtained and analyses of the data thus obtained without the necessity of human intervention. The NSS comprises of the following units such as

Unit 1—Subject with electrode headcap and sensors: This unit provides a means for seating the individual (subject) and a means for data collection with an electrode headcap, with EEG, EMG and respiration sensors to collect various bioelectric information. This bioelectric information is further transmitted to Unit 2

Unit 2—NSS Headbox: The NSS headbox receives the weak and noisy bioelectric information from the subject and conditions it, by removing the noise and amplifying the signal for better detection. Further the NSS Headbox is the first point of data processing and data filtering in the entire system. It electrically isolates the subject from the rest of the environment to ensure artifact free brain signals.

Unit 3—NSS signal conditioning unit: The signal conditioning unit receives the bio electric information from Unit 2 and reconditions the signals using an array of Filters, and sends the conditioned signal to the main acquisition system (Unit 4). The signal conditioning unit is the most critical hardware component in the entire system. It ensures that the brain signals are captured and delivered to the analysis unit without distortion.

Unit 4—NSS signal acquisition and processing unit: The unit receives the signal from Unit 3 and digitizes it for storage and analysis. The unit performs functions such as controlling of Unit 5, deciding if the bio electric activity is free from movement related artifacts or external noise and sends signals to Unit 5 to present auditory and/or visual stimuli to the subject. Further the unit receives information from unit 5 about the start and stop of the stimuli. Also the unit stores information about the start and stop of the stimuli as “Event Markers”, denoting the start of an Event, or stop or an Event. The event marker information is stored with the bioelectric activity. The unit also receives a video feed from a camera monitoring the subject. The video monitoring systems locks into the subject and captures video related information such as facial expression, eye ball movement and skin condition. This information captured does not affect the brain activation, and is not directly related to it.

The signal processing unit of Unit 4 is responsible for the analysis of the data collected. It captures and stores brain signals in their raw form. On the raw data captured, the unit is capable of performing complex analytical procedures to extract brain activation information from the same. The brain activation information is time locked with the probes presented and the video data captured. The information thus obtained is utilized to pinpoint the availability of experiential knowledge in the subject's brain. All the above recorded and processed data is stored on a secure NSS storage station to form a basis for documentary evidence.

Unit 5—Visual and Auditory Stimulus Presentation Unit (VASP): The unit and its functioning have been described in detail earlier. The unit is responsible for presenting visual and/or auditory stimulus to the subject.

Unit 6—Printer: A printer is utilized to print out the results of the test. Also means are provided to store the results in an electronic media and in the NSS system which can at a desired instant of time can be retrieved, displayed or printed out.

Unit 7—Analysis System: An analysis station is provided in the NSS to analyse the data collected by Unit 4. The unit is similar to Unit 4 but lacks the data acquisition features. The unit provides scientists with plurality of software tools needed to analyse and understand the brain activation information. The unit has the ability to access and analyse historical data from the NSS system which helps the scientists fine tune their probes and data extraction techniques.

Unit 8—Temperature and humidity sensing system: The unit has means for being connected to Unit 4. The unit has unique sensing mechanisms to monitor and record temperature and humidity and sends the information to assist Unit 4 to enable it make decisions with regards to its functioning in the current environment as determined by Unit 8.

Unit 9—Power Conditioning Unit: The unit protects the system from electrical abnormalities and provides clean low noise electricity to power the system.

The complete NSS system is electrically isolated from the rest of the environment. Means are provided to interface the various units of the NSS system to ensure smooth signal transfer between various units and to avoid human interference. The entire system is designed uniquely to automate the process of detection, processing and analysis of brain signatures and provides tamper proof results.

Exemplary data acquisition: Unit 4 of the NSS is responsible for data acquisition and analysis which have been described earlier. Acquisition of electrical oscillations commonly referred to as Electroencephalogram (EEG), from the scalp requires a plurality of electrodes. In a preferred embodiment of the present invention the system utilizes 30 such electrodes. Electronic amplifiers for amplification of weak signals in the range 15K to 20K and computer for signal storage are also essential for data acquisition. The amplifiers use a band filter for conditioning the signals so that frequencies within a certain range alone are amplified. However, there are many electrical artifacts produced by eye movements, neck and head movements, body movements, limb movements, respiratory changes and consciously produced mental activities. These are captured along with the EEG. For forensic purposes, the EEG must be artifact free as the interpretations are based on the accurate measurements of various frequencies and correcting the artifacts can confound these frequencies and their measurements. Further it is required that each probe is presented only when the online EEG is analysed and its integrity is found within acceptable limits. This is an unique feature of NSS which is not present in any other system. The online analysis facility finds out the mental status of the subject and prepares the probe for presentation. Presence of electrical oscillation supporting the following mental activity delays the presentation of the probe until predefined favorable condition is attained. These are
(a) The subject is mentally processing another activity such as praying, meditating.
(b) The subject is mentally thinking of another visual imagery
(c) The subject is avoiding listening to the probe by dozing or sleeping
(d) The subject has taken amphetamines or other drugs, which can interfere with his listening and thinking ability.

Percentages of electrical oscillations in different frequencies allow the system to identify the above conditions and delay or facilitate probe presentation. The following conditions are preset for such identification:

Percentage of 0-4 Hz oscillations in the baseline=Greater than 30% 1.

Percentage of 4-8 Hz oscillations in the baseline=Greater than 25% 2.

Percentage of 10-14 Hz oscillations in the baseline=Greater than 20% 3.

Percentage of 14-35 Hz oscillations in the baseline=Greater than 25% 4.

The program looks for the above activities at regular intervals and records the percentage on a visual chart for the experimenter's observation. The chart can also be viewed as brain maps for easy detection using a color scale of percentages.

In a preferred embodiment of the present invention, the continuously acquired Bioelectric oscillations from 30 cephalic, 2 eye movement channels and 2 Electromyography (EMG) channels are automatically detected, digitized and converted into a probe wise data file with details of the probes and their codes for further analyses. The 30 cephalic channels provide information about the bio-electrical oscillations over the left and right frontal, central, temporal and parieto-occipital regions. The analysis system divides the oscillations into segments of preset duration and identifies the segments of the pre-probe baseline and the response sections.

The electrical activities in the different channels are automatically analysed for each probe. Changes in each channel in each response segment due to the probe are statistically analysed and interpreted according to the specially developed algorithm for interpreting the presence of sensory registration of the probe, its semantic interpretation, and remembrance of experiential knowledge, emotional arousal and familiarity. The technology for detection of experiential knowledge of the act specified in the probe in a subject is based on the analyses of the energy levels, time domain changes and frequency changes of the electrical oscillations recorded from the different topographical areas of the brain.

Signal processing for computing the energy levels in different frequency ranges of the oscillations over the preset duration, stored in the hardware is carried out. Multiple statistical comparisons of the response segments stored in the hardware in each frequency range are carried out automatically. The analogue results of statistical comparisons in frequency in the multiple response segments, for each probe are converted to digital data for further interpretation by the system.

Statistically significant time domain changes are separately detected using the preset variables. Comprehensive interpretation of the digital results and the time domain changes are made by the system for the presence and level of cognitive processing. A final forensic report is prepared by the system and the report is printed out with the details of the controls used for data acquisition and analyses, and the interpretation of each probe. The signal processing system includes Multivariate analysis of variance and other linear parametric, so that the changes in the signal parameters derived through analyses in each trial are analysed independently and the results are interpreted according to the profile specifically developed for the system.

The neuro signature thus obtained can be repeatedly extracted from a person as long as the probe meaning does not change. Within the forensic domain, acquisition of experiential knowledge depends only on factors such as that the act was carried out in full consciousness, that is the subject was aware of every component of action executed. Experiential encoding may be defective if the person was in altered state of consciousness, or under alcohol or drug intoxication. Another important feature of the procedure is that the subject is not expected to make any response to the probes, except instructions to listen to them and recall as many as one remembers once the study is completed.

The NSS analysis utilizes primarily an oscillation analysis protocol and not time domain or Event Related Potential (ERP) analysis. Time domain analyses are also used for measuring morphologically predefined positive and negative deflections and use them in support of frequency analysis protocol. The analysis also employs an unique algorithm for identification of presence of neuro signatures if provoked by a probe. Further the present invention apart from its application in forensic investigation, the details of which were described earlier has been advantageously utilized in other areas of cognitive neuroscience such as in Clinical. Diagnosis where impairment of remembrance has critical importance as a preclinical symptom in various types of degenerative disorders producing dementia. Impairment of remembrance of recent experiential events of life is a frequently encountered problem in traumatic brain conditions. The technology of the present invention can be advantageously utilized to understand the recency of impairment of remembrance.

The invention described herein and as illustrated by the examples provides an efficient and integrated electronic investigative device for identification of truth that provides tamper proof bio-electric signatures unique to the subject.

The foregoing description of the invention has been set for merely to illustrate the invention and is not intended to be limiting. Since modifications of the disclosed embodiments incorporating the spirit and substance of the invention may occur to person skilled in the art, the invention should be construed to include everything within the scope of the appended claims and equivalents thereof.

I claim:

1. An electronic investigative device for finding the truth comprising of
At least one bio signal conditioning unit
Signal acquisition, Processing and Analysis Unit
Visual and Auditory Presentation Control Unit
Analysis Unit
Power Conditioning Unit
Optionally Temperature and Humidity Control Units and
Means for Printing the output from the analysis unit

2. A electronic investigative device of claim 1, wherein said system comprises of at least one Bio signal conditioning unit that
receives the bio electric information from the subject and reconditions the signals and wherein said reconditioning of the signal is achieved by means of an array of Filters and said signal is digitized for storage and analysis.

3. A electronic investigative device of claim 1, wherein said system comprises of signal acquisition and processing unit provided with means to
receive information about the start and stop of the stimuli;
store information about the start and stop of the stimuli denoting the start of an Event, or stop of an Event and
having means for providing tamper proof neuro signatures.

4. A electronic investigative device of claim 1, wherein said signal processing unit comprises of video monitoring system that locks into the subject and captures video related information such as facial expression, eye ball movement and skin condition.

5. A electronic investigative device of claim 1, wherein the said system comprises of a Visual and Auditory Stimulus Presentation Unit (VASP) wherein the said unit is utilized for presenting visual and/or auditory stimulus to the subject.

6. A novel method of administering truth finding tests employing the device claimed in claim 1, wherein the said method comprises of
Collation of information
Validation of the probes
Creation of nestled probes
Presentation of the probes and
Detection of bio electric responses from brain.

7. A method of claim 6, wherein the said collation of information comprises of identification and categorization of at least one auditory and/or one visual event experienced by the subject and wherein the said collation of said at least one auditory and/or visual event constitutes a probe.

8. A probe of claim 7, wherein the said probe can be categorized into categories such as
Neutral probes that comprises of at least one auditory stimulus of generic and/or semantic nature unconnected to the test;
Control probes that comprises of auditory and/or visual stimulus related to the personal life events of the subject which are utilized to validate the findings of the target probes and
Target probes that comprises of auditory and/or visual stimulus that are responsible for detection of truth
And collation of Neutral, Control and Target probe constitute a nestled probe.

9. A method of claim 6, wherein a plurality of the said nestled probe maybe presented to the subject for detection of neuro signatures.

10. A method of claim 6, wherein the said presentation of the said probes is achieved by the visual and auditory stimulus presentation unit wherein such a unit is provided with means for control of the probe presentation to the subject; synchronization of probe presentation; and enablement of visualization of the probes presented.

11. A method of claim 6, wherein the said detection of neuro signatures by the said presentation of probes is utilized to generate real time bio electric responses.

12. A method of claim 6, wherein the detection of the said neuro signatures is unique to the presentation of the probes and/or unique to the subject.

13. An electronic investigative device as substantially herein described and as illustrated in the accompanying drawings.

14. A method of administering truth-finding tests employing the device as substantially herein described and as illustrated in the accompanying drawings.
